(19) United States
(12) Patent Application Publication
(10) Pub. No.: US 2017/0076826 A1
(43) Pub. Date: Mar. 16, 2017

(54) RADIATION SHIELD APRON WITH IMPROVED GONADAL PROTECTION

(71) Applicant: Svetlana Karasina, Basking Ridge, NJ (US)

(72) Inventor: Svetlana Karasina, Basking Ridge, NJ (US)

(21) Appl. No.: 15/264,522

(22) Filed: Sep. 13, 2016

Related U.S. Application Data
(60) Provisional application No. 62/217,978, filed on Sep. 14, 2015.

(51) Int. Cl. G21F 3/03; G21F 1/08; A61B 6/10; A41D 13/04; A41D 13/12

(52) U.S. Cl. CPC G21F 3/03; A41D 13/04; A41D 13/1209; A61B 6/107; G21F 1/085

(57) ABSTRACT

A protective garment comprising gonadal-abdominal protective section attached to a shield apron, the gonadal-abdominal protective section comprising front cover sheet, back cover sheet, one or more inner sheets of flexible radiation shielding material arranged between the front cover sheet and the back cover sheet, and an upper edge attached to a bottom portion of the shield apron.

### CROSS REFERENCE TO RELATED APPLICATION

This application claims the priority of U.S. Provisional Application No. 62/217,978, entitled “RADIATION SHIELD APRON WITH IMPROVED GONADAL PROTECTION,” filed on Sep. 14, 2015, the disclosure of which is hereby incorporated by reference in its entirely.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND OF THE INVENTION

Field of the Invention

The present application relates to an apron-like garment for protecting the wearer of said garment from harmful X-rays and other radiation.

Description of the Prior Art

Radiation shield aprons are known to be used by medical professionals who normally assume a sitting position while exposed to radiation. However, all known shield aprons commonly comprise essentially rectangular front panel which extends from the tops of the shoulders and base of the neck to at least about knee level of the wearer where side edges of the front panel are parallel to each other and where a bottom edge of the front panel is essentially perpendicular to the side edges of the front panel. The described general scheme of the existing protective aprons is associated with a serious flow of failing to adequately protect gonadal area of a wearer because the bottom edge of the existing protective aprons, due to its rigidness (as having a lead insert within) and due to its essential rectangular configuration tends to lay horizontally flat on the wearer's laps without bending over the wearer's laps sufficiently enough to protect the gonadal area of the wearer.

### SUMMARY OF THE INVENTION

The present invention provides a protective garment comprising gonadal-abdominal protective section attached to a shield apron, the gonadal-abdominal protective section comprising front cover sheet, back cover sheet, one or more inner sheets of flexible radiation shielding material arranged between the front cover sheet and the back cover sheet, and an upper edge attached to a bottom portion of the shield apron.

The front cover sheet and the back cover sheet may comprise at least one of cotton, nylon, rubber, vinyl, plastics, or synthetic fibers. The flexible radiation shielding material can be comprised of heavy-metal metals such as lead, tin, antimony, tungsten, or bismuth. According to one embodiment, the front cover sheet or the back cover sheet may be coated with bismuth oxide.

The gonadal-abdominal protective section further comprises a lower edge. The upper edge and the lower edge of the gonadal-abdominal protective section comprises an upwardly radiating semi-circular shape. In one embodiment, the upper edge is straightened into a substantially horizontal edge. The upper edge may be attached to the bottom portion of the shield apron by at least one of stitching, buttons, zipper, clips, and hook and loop fasteners.

### BRIEF DESCRIPTION OF THE DRAWINGS

The invention is illustrated in the figures of the accompanying drawings which are meant to be exemplary and not limiting, in which like references are intended to refer to like or corresponding parts, and in which:

FIG. 1 illustrates a front view of an improved gonadal-abdominal protective section according to an embodiment of the present invention;

FIG. 2 illustrates a front view of an improved gonadal-abdominal protective section in a modified configuration prior to its attachment to a front panel of a shield apron according to an embodiment of the present invention;

FIG. 3 illustrates a front view of a wearer in a standing up position and wearing a shield apron having an improved gonadal-abdominal protective section according to an embodiment of the present invention;

FIG. 4 illustrates a front view of a wearer in a sitting position and wearing a shield apron having an improved gonadal-abdominal protective section according to an embodiment of the present invention; and

FIG. 5 illustrates a cross-section view of improved gonadal-abdominal protective section according to an embodiment of the present invention.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. It is to be understood that other embodiments may be utilized and structural changes may be made without departing from the scope of the present invention. Likewise, a reasonably broad scope for claimed or covered subject matter is intended. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of exemplary embodiments in whole or in part.

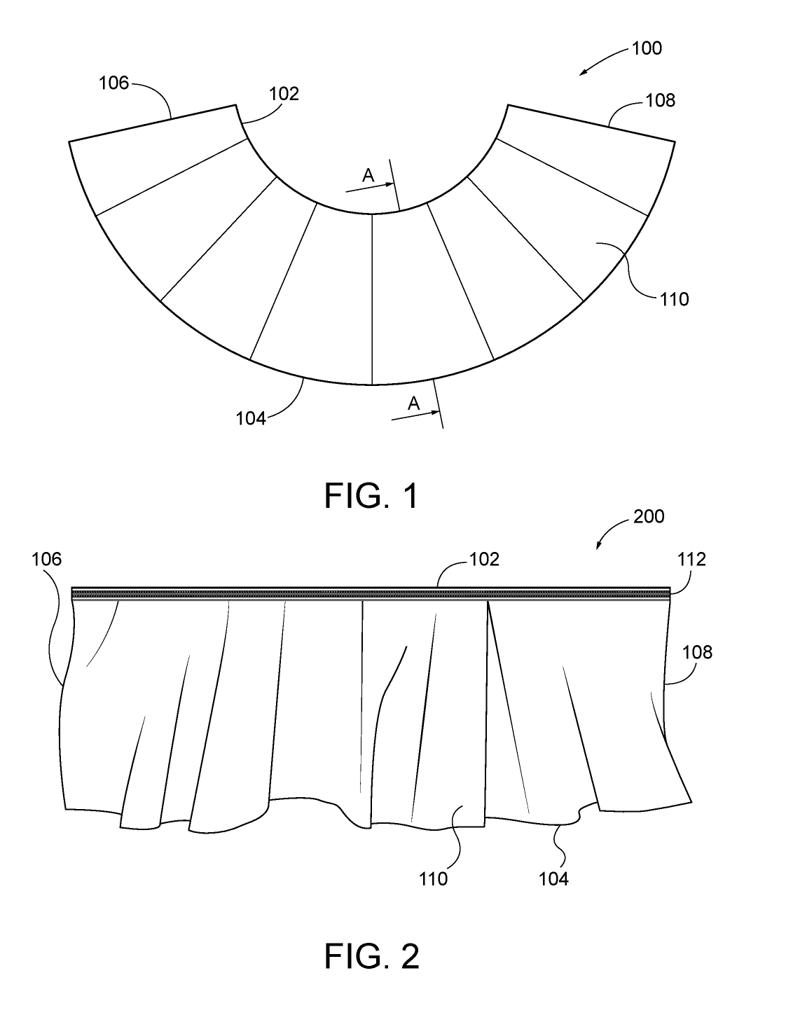

In FIG. 1, an improved gonadal-abdominal protective section 100 for integration with a shield apron is presented in the form of U-shaped high-strength fabric having a front cover sheet and back cover sheet and one or more inner sheets of flexible radiation shielding material enclosed between the front cover sheet and back cover sheet. Front cover sheet and back cover sheet may be constructed from fabrics such as cotton, nylon, rubber, vinyl, plastics, or synthetic fibers. Flexible radiation shielding material 110 may be comprised of heavy metal materials such as lead, tin, antimony, tungsten, bismuth, other heavy materials that attenuate radiation, and composites thereof. Optionally, the fabric of the front and back cover sheets may be coated with bismuth oxide or other radiation shielding coatings.

The U-shape of the improved gonadal-abdominal protective section 100 comprises an upper edge 102, lower edge 104, and two straight side edges 106 and 108, where the upper edge 102 is substantially parallel to the lower edge 104 and where one straight side edge 106 is not parallel to one other straight side edge 108. As illustrated, upper edge 102 and lower edge 104 comprise an upwardly radiating semi-circular shape when in a relaxed or unstressed configuration. It is to be understood that a degree of radius of the upper edge 102 and of the lower edge 104 may be identical or it may differ. The metrics of radius of the upper edge 102 and of the lower edge 104 is non-limiting variable and may be changed depending on the measurements of the wearer.

FIG. 2 presents the improved gonadal-abdominal protective section 100 in the form to be attached to the shield apron. As may be readily appreciated from FIG. 2, the upper edge 102 of the improved gonadal-abdominal protective section 100 is straightened such that the upper edge 102 is substantially horizontal. Straightening the upper edge 102 into a straight horizontal edge causes the two straight side edges 106 and 108 to be substantially parallel to each other while the mid-section 110 of the improved gonadal-abdominal protective section 100 is distorted or compressed as shown in FIG. 2.

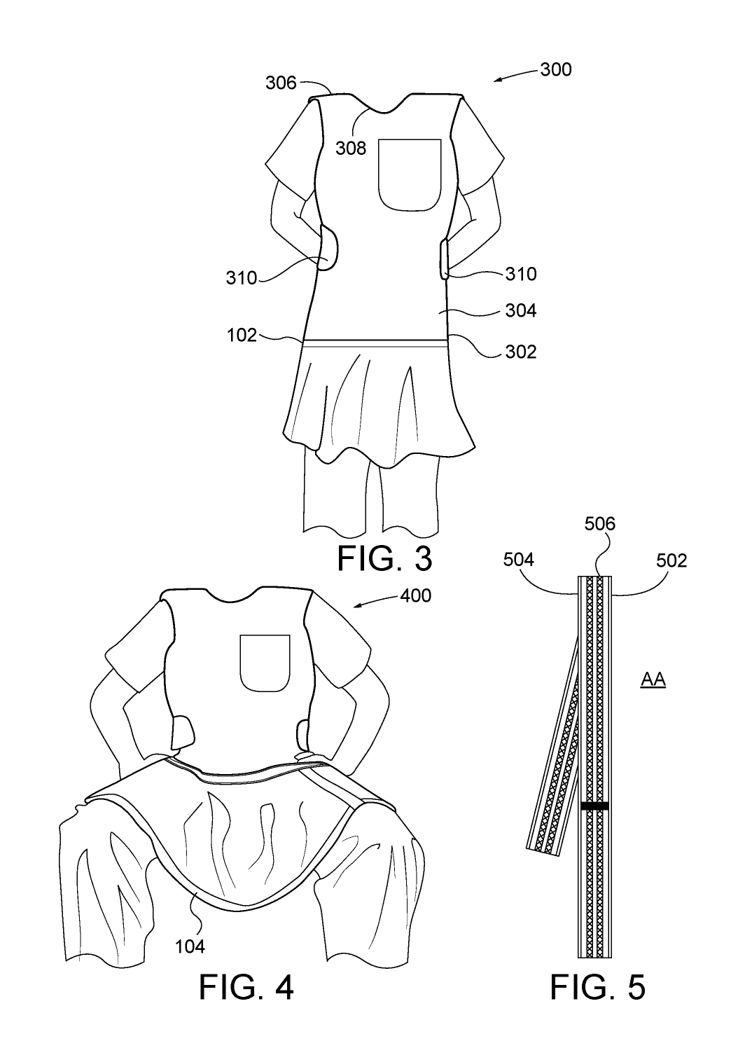

The improved gonadal-abdominal protective section 100, as illustrated in FIGS. 1 and 2, may be attached to a bottom portion 302 of a shield apron 300 as shown in FIG. 3. A shield apron 300 comprises a front shield panel 304 and a pair of separate back wing panels 310 (partially illustrated). A front shield panel 304 extends downwardly from the tops of the shoulders 306 and base of the neck 308 to at least about half-thigh level of the wearer and from side to side across the front and around the opposite sides of the wearer. The bottom portion 302 can be seamlessly sewn to or physically attached to upper edge 102 of the improved gonadal-abdominal protective section 100 in such a way that lower edge of the bottom portion 302 of the shield apron 300 and the top portion of the upper edge 102 are overlapped to ensure sufficient protection of the stitching from exposure to radiation. According to certain embodiments, upper edge 102 of improved gonadal-abdominal protective section 100 may be attached with the bottom portion 302 of shield apron 300 by means such as, but not limited to, buttons, zippers, clips, hook and loop fasteners, and etc.

FIG. 3 presents a view of the shield apron 300 having the improved gonadal-abdominal protective section 100 when the wearer is in his standing up position while the improved gonadal-abdominal protective section 100 is shown to maintain its secure coverage of the gonadal area of the wearer.

FIG. 4 presents a view of the shield apron 400 having the improved gonadal-abdominal protective section 100 when the wearer is in his sitting position while the improved gonadal-abdominal protective section 100 is shown to securely cover the gonadal area of the wearer ensuring that the lower edge 104 of the improved gonadal-abdominal protective section 100 is positioned in such a way as to protect gonadal area of the wearer. Specifically, because of the profile design of the improved gonadal-abdominal protective section 100, it causes the improved gonadal-abdominal protective section to be close to the body of the wearer throughout, as shown in FIG. 4.

FIG. 5 presents a cross-section view AA of the improved gonadal-abdominal protective section 100, as presented in FIGS. 1-4, having a front cover sheet 502 and back cover sheet 504 and one or more inner sheets of flexible radiation shielding material 506 enclosed between the front cover sheet 502 and back cover sheet 504.

FIGS. 1 through 5 are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present invention encompasses present and future known equivalents to the known components referred to herein by way of illustration.

The foregoing description of the specific embodiments will so fully reveal the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

What is claimed is:

1. A protective garment comprising:
gonadal protective section attached to a shield apron, the gonadal protective section comprising:
front cover sheet;
back cover sheet;
one or more inner sheets of flexible radiation shielding material arranged between the front cover sheet and the back cover sheet; and
an upper edge attached to a bottom portion of the shield apron.

2. The protective garment of claim 1 wherein the front cover sheet and the back cover sheet comprise at least one of cotton, nylon, rubber, vinyl, plastics, or synthetic fibers.

3. The protective garment of claim 1 wherein the flexible radiation shielding material is selected from a group consisting of lead, tin, antimony, tungsten, and bismuth.

4. The protective garment of claim 1 wherein the front cover sheet or the back cover sheet is coated with bismuth oxide.

5. The protective garment of claim 1 wherein the gonadal-abdominal protective section further comprises a lower edge.

6. The protective garment of claim 5 wherein the upper edge and the lower edge comprises an upwardly radiating semi-circular shape.

7. The protective garment of claim 5, wherein the upper edge and the lower edge are parallel to each other.

8. The protective garment of claim 5, wherein the upper edge and the lower edge are not parallel to each other.

9. The protective garment of claim 5, wherein the upper edge is straightened into a substantially horizontal edge upon attachment to the bottom portion of the shield apron.

10. The protective garment of claim 1, wherein the wrap-around waist band comprises an elastic that is drawn around a wearer's waist.

11. The protective garment of claim 1, wherein the upper edge is attached to the bottom portion of the shield apron by at least one of buttons, zipper, clips, and hook and loop fasteners.

12. The protective garment of claim 1, wherein the upper edge comprises a wrap-around waist band.
